(19) United States
(12) Patent Application Publication
(10) Pub. No.: US 2019/0119729 A1
(43) Pub. Date: Apr. 25, 2019

(54) METHOD OF DETECTING AND TREATING P. ACNES AND KIT THEREOF

(71) Applicant: VYOME BIOSCIENCES PVT. LTD., Delhi (IN)

(72) Inventors: Shiladitya SENGUPTA, Cambridge, MA (US); Shamilk GHOSH, Uttar Pradesh (IN); Mau SINHA, Uttar Pradesh (IN); Suresh SADHASIVAM, Tamil Nadu (IN); Anamika BHATTACHARYYA, Delhi (IN)

(21) Appl. No.: 16/070,416

(22) PCT Filed: Jan. 20, 2017
(86) PCT No.: PCT/IB2017/050313

§ 371 Date: Jan. 15, 2019

(30) Foreign Application Priority Data
Jan. 22, 2016 (IN) 201611002481

(51) Int. Cl. C12Q 1/689; A61K 31/65; A61K 31/192; A61K 31/4745; A61K 45/06; A61P 17/10

(52) U.S. Cl. CPC C12Q 1/689; A61K 31/65; A61K 31/192; C12Q 2600/106; A61K 45/06; A61P 17/10; C12Q 2600/156; A61K 31/4745

(57) ABSTRACT

The present disclosure relates to method for detecting P. acnes as well as a method for detecting mutation in genomic sequence of 23S rRNA sequence of P. acnes, particularly mutation A2058G. The method comprises amplifying a region of 23S rRNA specific for P. acnes using primers of SEQ ID Nos. 1 and 2 followed by detecting the mutation through selective action of an endonuclease- at a mismatch at the site of said mutation or post-hybridization with specific P. acnes 23S regions followed by mismatch specific endonuclease action. The presence of A2058G mutation confers antibiotic resistance, particularly clindamycin and erythromycin resistance, and thus the present disclosure also relates to methods of treating acne caused by clindamycin resistant P. acnes by using fluoroquinolone based antibiotics.

### TECHNICAL FIELD

The present disclosure relates to method for detecting P. acnes. The present disclosure also relates to method(s) for detecting antibiotic resistant or sensitive strains of P. acnes by detecting point mutation in the 23S rRNA genomic sequence of P. acnes. The present disclosure also relates to suitable treatment regimens for patients based on the strains of P. acnes they are infected with.

### BACKGROUND OF THE DISCLOSURE

Acne vulgaris affects almost 9.4% of the world's population, making it the 8th most prevalent disease (Tan & Bhate 2.015, Br l Dermatol 172: 3). While acne is known to be a multifactorial disease, the key etiological factor in the development of acne is Propionibacterium acnes (P. acnes), a Gram-positive rod, which is present as a part of the natural microflora of the skin (Cunliffe et al 1981 Clin Exp Dermatol 6: 461; Holland et al 1981 J Appl Bacteriol 51: 195).

Antibiotics are an integral part of acne treatment, not only due to their antibiotic effect but also by their anti-inflammatory action. Antibiotics are recommended as part of the first choice of treatment for mild-to-moderate, mixed, papular/pustular, and moderate nodular acne, and as an alternative choice in severe nodular/conglobate acne. Antibiotics act on specific bacterial targets leading to the decrease in bacterial load in the sebaceous follicles of patients with acne. Topical antibiotics mostly prescribed for acne include erythromycin (macrolide class) and clindamycin (lincosamide class).

However, indiscriminate use of antibiotics has raised the concern of generation of resistant strains of P. acnes which can lead to treatment failure of the currently prescribed antibiotics. Combined resistance to erythromycin and clindamycin in cutaneous P. acnes was first reported in 1979 in the US in 20% of acne patients using topical formulations of either drug (Crawford et al 1979 J Invest Dermatol 72: 187). The antibiotic sensitivities of different P. acnes isolates reported in different studies worldwide against the common antibiotics depict alarming resistance rates of erythromycin and clindamycin from U.S.A., Europe, and Asia. Resistance to clindamycin has evolved to a staggering ⅓^{rd} of patients in the US carrying resistant strains of P. acnes (Ross et al 2041 Br J Dermatol 144: 339). A study revealed a >90% incidence of clindamycin-resistant P. acnes strains in Spain (Ross et al 2003 Br J Dermatol 148: 467). Similarly, the average MIC for erythromycin was found to increase 100-fold in those receiving prolonged antibiotic therapy with the drug (Leyden et al 1983 J Am Acad Dermatol 8: 41).

Several investigations have indicated that presence of clindamycin resistant P. acnes strains may indeed manifest as therapeutic failure either a reduced response, no response, or relapse (Mills et al 2002 Acta Derm Venereol 82: 260). A study found that patients infected with resistant P. acnes strains had higher bacterial counts and poorer clinical outcomes than patients infected with sensitive strains (Leyden et al 1983 J Am Acad Dermatol 8: 41). A gradual decrease in the efficacy of topical erythromycin on inflammatory and noninflammatory lesions over time was observed in clinical trials of therapeutic intervention for acne, which is probably related to the development of antibiotic-resistant P. acnes (Simonart Dramaix. 2005 Br J Dermatol 153: 395). In a recent case study, a patient with clindamycin-resistant strains of P. acnes was found not to respond to conventional antibiotic treatment, but exhibited resolution of acne when the antibiotic regimen was changed (Sadhasivam et al 2016 Dermatol Ther 29: 451). These findings indicate that the emergence of resistance is indeed a driver for antibiotic-therapy failure in acne. However, in absence of a guiding test, patients are treated with the antibiotic even if they are already resistant to the drug. This means over-use of antibiotics, which can lead to cross-resistance in other therapeutic indications. There is an urgent need for a test that can allow the selection of patients for treatment with the right antibiotic.

Clindamycin exerts its antimicrobial effect by inhibiting bacterial protein synthesis by binding to the 23S rRNA of the 50S subunit. It interferes with the A site and P site substrate binding and physically hinders the path of the growing peptide chain. The clindamycin resistance of P. acnes is caused by point mutations in domain V of the 23S rRNA or by alteration of the target site by the 23S dimethylase encoded by erm(X). The mutation at 2058 (E. coli numbering) from A to G is very critical and results in high clindamycin-resistance in P. acnes compared to the other residues (Ross et al 1997 Antimicrob Agents Chemother 41: 1162; Ross et al 1998 Dermatology 196: 69). Several studies have reported A2058 as one of the pivotal nucleotides for lincosamide binding (Poehlsgaard et al 2005 Antimicrob Agents Chemother 49: 1553; Schlünzen et al 2001 Nature 413: 814). Thus, a mutation in this residue from A to G severely hampers the drug binding. Patients harboring resistant P. acnes strains with A.2058G mutations have shown therapeutic failure when prescribed clindamycin (Mills et al 2002 Acta Derm Venereol 82: 260).

The current combination therapy of using non-antibiotics like benzoyl peroxide or retinoids with clindamycin might be ineffective in patients already harboring clindamycin-resistant strains, as only the benzoyl peroxide or retinoid component of the combination would be active in these patients. A knowledge about the presence of P. acnes with clindamycin resistance-conferring mutations can help screen clindamycin non-responders, and thereby reduce the indiscriminate use of this antibiotic. However, currently, the choice of antibiotics in the management of acne is random, and patients are being treated with common antibiotics, mostly clindamycin, without any genetic insights on whether the bacteria will respond to the treatment or not. Further, routine P. acnes isolation, identification and antibiotic susceptibility testing takes ten to twenty days to arrive to conclusion whether a patient is infected with drug resistant P. acnes. Also, currently available literature for detecting mutations in the clindamycin resistance conferring region (23S rRNA) of bacteria report first identifying the organism by amplifying a region specific to that organism and then using a second method (PCR, sequencing or mutation specific probe hybridization techniques) to detect the specific mutation in the 23S rRNA region, where the region containing the mutation is different from the region amplified for identifying the organism (J. Med. Microbiol. 63(5):721-728 doi:10. 1099/jmm.0.067611-0; Microbiology and Immunology, 52: 621-624). Further, currently, detection of resistance-conferring mutations by sequencing, hybridization with probes, use of amplification primers, etc. involve initial culturing and isolation of P. acnes from the sample, prior to the detection step. Therefore, these techniques are time consuming and costly.

Thus, there is a need for quick methodologies to evaluate the presence of P. acnes clindamycin and erythromycin resistance. This allows both an adequate management of acne patients who need antibiotic treatment and monitoring of the epidemiological evolution of bacterial resistance. Tailoring treatment, where a patient is treated with an antibiotic based on the absence of a resistance-conferring mutation, will be a paradigm shift in the management of acne.

### SUMMARY OF THE DISCLOSURE

Accordingly, the present disclosure relates to a method for detecting Propionibacterium acnes (P. acnes) in a sample, said method comprising contacting the sample with forward primer set forth in SEQ ID No. 1 and reverse primer set forth in SEQ ID No. 2 or with forward primer set forth in SEQ ID No. 7 and reverse primer set forth in SEQ ID No. 8 and subjecting to polymerase chain reaction (PCR) to obtain an amplified product, wherein obtaining the amplified product confirms presence of P. acnes in the sample; a method for detecting mutation in 23S rRNA sequence of P. acnes, said method comprising acts of: (a) contacting DNA encoding the 23S rRNA sequence of P. acnes with primers specific for P. acnes 23S rRNA sequence, wherein forward primer is set forth in SEQ ID No. 1 and reverse primer is set forth in SEQ ID No. 2, or wherein forward primer is set forth in SEQ ID No. 7 and reverse primer is set forth in SEQ ID No.8, (b) subjecting the contacted sequence of P. acnes to polymerase chain reaction (PCR) to obtain amplified product, and (c) subjecting the amplified product to restriction enzyme digestion, or DNA hybridization followed by endonuclease digestion, for detecting the mutation; a method of treating acne in a patient infected with P. acnes, said method comprising acts of: (a) obtaining sample from a patient, (b) detecting presence or absence of mutation in P. acnes to determine whether said P. acnes is resistant or sensitive to an antibiotic A; and (c) administering the patient with an antibiotic B if the P. acnes is antibiotic resistant to the antibiotic A; and a kit for detecting and optionally treating P. acnes, said kit comprising primer sequences of SEQ ID Nos. 1 and 2 or primer sequences of SEQ ID Nos. 7 and 8, or both, optionally along with dNTPs, DNA polymerase, buffer, nuclease free water, magnesium chloride, vials, restriction endonuclease, DNA endonuclease, P. acnes wild type strain, DNA encoding 23S rRNA of P. acnes containing A2058G mutation, DNA encoding 23S rRNA of P. acnes, containing A2059G mutation, DNA encoding 23S rRNA of P. acnes containing A2058G and A2059G mutation, instruction manual, and an antibiotic, or any combination thereof.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure where:

FIG. 1(a) shows the 23S rRNA sequence of wild type P. acnes, FIG. 1(b) shows the 23S rRNA sequence of P. acnes strain carrying the A2058G mutation (E. coli numbering) and FIG. 1(c) shows the 23S rRNA sequence of P. acnes strain carrying the A2059G mutation (E. coli numbering).

FIG. 2 is a flowchart depicting the sequence of steps involved in the method of diagnosis of antibiotic resistance of P. acnes according to the present disclosure.

FIGS. 3(a) and 3(b) depict the action of restriction endonuclease in detecting P. acnes (wild type) 23S rRNA and P. acnes (mutated-A2058G) 23S rRNA.

FIG. 4(a) depicts agarose gel showing amplification profile of the genomic sequence of 23S rRNA using primers of SEQ ID Nos. 1 and 2 from different strains of P. acnes, wherein Lane 1: MTCC 1951, Lane 2: CCARM 9010. Lane 3: MTCC 3297, Lane 4: V21A6, Lane 5: V21A7, Lane 6: V21B2, Lane 7; Marker (100 bp ladder),

FIG. 4(b) depicts agarose gel showing the amplification profile of the genomic sequence of 23S rRNA using primers of SEQ ID Nos. 1 and 2 from different bacteria, wherein Lane 1: S. epidermidis M3-1, Lane 2: S. epidermidis M4-1, Lane 3: P. avidum S15A-1, Lane 4: P. avidum S3-1, Lane 5: P. acnes CCARM 9010, Lane 6: P. acnes MTCC 1951, Lane 7: Water, Lane 8: Marker (100 bp ladder).

FIG. 4(c) depicts agarose gel showing the amplification profile of the genomic sequence of 23S rRNA using primers of SEQ ID Nos. 1 and 2 with mixed cultures, wherein Lane 1: Marker (100 bp ladder), Lane 2: Mixed Culture 1 (with P. acnes), Lane 3: Mixed Culture 2 (without P. acnes), Lane 4: Water.

FIG. 5(a) is a flow chart depicting the action of mismatch specific endonuclease on amplified samples hybridized (denatured and re-annealed) with supplied template DNA for detecting P. acnes (wild type) 23S rRNA, P. acnes (A2058G) 23S rRNA and P. acnes (A2059G) 23S rRNA in unknown samples.

FIG. 5(b) depicts agarose gel showing the products obtained post digestion with mismatch specific restriction endonuclease, wherein Lane 1: Marker, Lane 2: MTCC 1951+ CCARM 9010 (10 μl each)-Nuclease S-3 μl, Lane 3: MTCC 1951+ CCARM 9010 (10 μl each)-Nuclease S-3 μl, Lane 4: MTCC 1951+MTCC 1951(10 μl each)-Nuclease S-3 μl, Lane 5: Positive Control C+G (10 μl each), Lane 6: Negative Control C+C (10 μl each).

FIG. 6 is a graph showing reduction in bacterial count with besifloxacin topical formulation in P. acnes skin infection in mouse.

### DETAILED DESCRIPTION OF THE DISCLOSURE

The present disclosure relates to a method for detecting Propionibacterium acnes (P. acnes) in a sample, said method comprising contacting the sample with forward primer set forth in SEQ ID No. 1 and reverse primer set forth in SEQ ID No. 2 or with forward primer set forth in SEQ ID No. 7 and reverse primer set forth in SEQ ID No. 8 and subjecting to polymerase chain reaction (PCR) to obtain an amplified product, wherein obtaining the amplified product confirms presence of P. acnes in the sample.

In an embodiment of the present disclosure, the primers specifically amplify DNA encoding 23S rRNA sequence of P. acnes set forth in SEQ ID No. 3, SEQ ID No. 4, SEQ ID No. 5 or SEQ ID No. 6.

In another embodiment of the present disclosure, the SEQ ID No. 4 possesses mutation at position 2058 (E. coli numbering) with respect to wild type SEQ ID No. 3 and the mutation is A2058G; the SEQ ID No. 5 possesses mutation at position 2059 (E. coli numbering) with respect to wild type SEQ ID No. 3 and the mutation is A2059G; and the SEQ ID No. 6 possesses mutation at positions 2058 and 2059 (E. coli numbering) with respect to wild type SEQ ID No. 3 and the mutations are A2058G and A2059G.

In yet another embodiment of the present disclosure, the sample is selected from group comprising water, food, environmental sample and biological sample.

The present disclosure also relates to a method for detecting mutation in 23S rRNA sequence of P. acnes, said method comprising acts of:
(a) contacting DNA encoding the 23S rRNA sequence of P. acnes with primers specific for P. acnes 23S rRNA sequence, wherein forward primer is set forth in SEQ ID No. 1 and reverse primer is set forth in SEQ ID No. 2, or wherein forward primer is set forth in SEQ ID No. 7 and reverse primer is set forth in SEQ ID No.8;
(b) subjecting the contacted sequence of P. acnes to polymerase chain reaction (PCR) to obtain amplified product; and
(c) subjecting the amplified product to restriction enzyme digestion, or DNA hybridization followed by endonuclease digestion, for detecting the mutation.

In an embodiment of the present disclosure, the PCR amplification is carried out in presence of amplification reagent selected from group comprising dNTP, magnesium chloride, buffer, nuclease free water and DNA polymerase, or any combination thereof.

In another embodiment of the present disclosure, the detection of mutation by restriction enzyme digestion comprises acts of:
(a) contacting the amplified product with restriction enzyme having affinity for the mutation to be detected, to enable cleaving of the amplified product at point of mutation; and
(b) detecting the mutation by determining presence of cleaved amplified product post subjecting the amplified product to restriction enzyme digestion.

In yet another embodiment of the present disclosure, the restriction enzyme is BpuJI.

In yet another embodiment of the present disclosure, the detection of mutation by DNA hybridization followed by endonuclease digestion comprises acts of:
(a) contacting one of the amplified products with DNA encoding 23S rRNA sequence of wild type P. acnes or with DNA encoding 23S rRNA sequence of P. acnes containing the specific mutation to be detected, or contacting the amplified products individually with both; and subjecting to hybridization to obtain a duplex;
(b) contacting the duplex with a DNA endonuclease; and
(c) detecting the mutation by determining presence or absence of cleaved duplex at point of mutation, post subjecting the duplex to endonuclease digestion.

In yet another embodiment of the present disclosure, when the amplified product has a mutation, hybridization with the DNA encoding 23S rRNA sequence of wild type P. acnes results in a cleaved duplex post contacting with the DNA endonuclease; when the amplified product has a mutation, hybridization with the DNA encoding 23S rRNA sequence of P. acnes containing the specific mutation results in an un-cleaved duplex post contacting with the DNA endonuclease; or when the amplified product has no mutation, hybridization with the DNA encoding 23S rRNA sequence of wild type P. acnes results in an un-cleaved duplex post contacting with the DNA endonuclease; or when the amplified product has no mutation or a mutation other than the mutation present in the sequence it is hybridized with, hybridization with the DNA encoding 23S rRNA sequence of P. acnes containing the specific mutation results in a cleaved duplex post contacting with the DNA endonuclease; and wherein the DNA hybridization is carried out by melting the contacted amplified product at temperature ranging from about 90° C. to about 95° C. for time period ranging from about 5 to about 10 minutes to obtain melted strands, followed by reannealing the melted strands by cooling to a temperature ranging from about 20° C. to about 27° C. to obtain the duplex; and wherein the DNA endonuclease is a mismatch specific endonuclease selected from group comprising CELII, CELI and T7 endonuclease.

In yet another embodiment of the present disclosure, determination of amplified product, cleaved amplified product or cleaved duplex is carried out using Agarose gel electrophoresis.

In yet another embodiment of the present disclosure, the P. acnes is present in a sample selected from group comprising water, food, environmental sample and biological sample.

In yet another embodiment of the present disclosure, the mutation in the 23S rRNA of P. acnes is selected from A2058G (E. coli numbering), A2059G (E. coli numbering) or a combination thereof.

In yet another embodiment of the present disclosure, the mutation is A2058G (E. coli numbering).

In still another embodiment of the present disclosure, when the mutation is A2058G or a combination of A2058G and A2059G, the P. acnes is rendered antibiotic resistant.

The present disclosure also relates to a method of treating acne in a patient infected with P. acnes, said method comprising acts of:
(a) obtaining sample from a patient;
(b) detecting presence or absence of mutation in P. acnes by the method as above to determine whether said P. acnes is resistant or sensitive to an antibiotic A; and
(c) administering the patient with an antibiotic B if the P. acnes is resistant to the antibiotic A.

In an embodiment of the present disclosure, the P. acnes is not isolated from the sample for detection.

In another embodiment of the present disclosure, the sample is a biological sample selected from group comprising body fluid, tissue, skin, acne lesion and bone, or any combination thereof, preferably acne lesion; and wherein the body fluid is blood.

In yet another embodiment of the present disclosure, the biological sample is acne lesion.

In yet another embodiment of the present disclosure, the antibiotic is lincosamide or macrolide or a combination thereof.

In yet another embodiment of the present disclosure, the antibiotic is clindamycin, erythromycin or a combination thereof.

In yet another embodiment of the present disclosure, the antibiotic A is lincosamide or macrolide or a combination thereof; and wherein the antibiotic B is a fluoroquinolone antibiotic selected from group comprising besifloxacin, nadifloxacin and levafloxacin.

In yet another embodiment of the present disclosure, the antibiotic A is clindamycin, erythromycin or a combination thereof.

In yet another embodiment of the present disclosure, the antibiotic B is present at concentration ranging from about 0.1% w/w to about 10% w/w; and wherein the antibiotic B is in form of a formulation selected from group comprising topical formulation, solid oral formulation, liquid oral formulation, inhalation formulation, nasal formulation, ophthalmic formulation, parenteral formulation, phytoceutical, nutraceutical and food stuff or any combination thereof.

In yet another embodiment of the present disclosure, the antibiotic B is present at concentration ranging from about 0.2% w/w to about 5% w/w. In a further embodiment, the antibiotic B is present at concentration ranging from about 1% w/w to about 2% w/w.

In yet another embodiment of the present disclosure, the antibiotic B is in form of a topical formulation.

In yet another embodiment of the present disclosure, the topical formulation is selected from group comprising cream, gel, spray, foam, lotion or any combination thereof; the solid oral formulation is selected from group comprising tablet, capsule, troche, lozenge, dispersible powder, dispersible granule or any combination thereof; the liquid oral formulation is selected from group comprising aqueous or oily suspension, emulsion, drop, emulsion in hard or soft gel capsule, syrup, elixir or any combination thereof; the parenteral formulation is selected from group comprising intravenous injection, intramuscular injection, intramuscular depot, subcutaneous injection, percutaneous injection or any combination thereof; the inhalation formulation is selected from group comprising inhaler, dry powder inhaler, nebulizer or any combination thereof; the nasal formulation is selected from group comprising nasal drops, nasal sprays or a combination thereof; and the ophthalmic formulation comprises eye drops.

In still another embodiment of the present disclosure, the formulation further comprises tetracycline, adapalene, benzoyl peroxide, or combinations thereof.

The present disclosure further relates to a kit for detecting and optionally treating P. acnes, said kit comprising primer sequences of SEQ ID Nos. 1 and 2 or primer sequences of SEQ ID Nos. 7 and 8, or both, optionally along with dNTPs, DNA polymerase, buffer, nuclease free water, magnesium chloride, vials, restriction endonuclease, DNA endonuclease, P. acnes wild type strain, DNA encoding 23S rRNA of P. acnes containing A2058G mutation, DNA encoding 23S rRNA of P. acnes containing A2059G mutation, DNA encoding 23S rRNA of P. acnes containing A2058G and A2059G mutation, instruction manual, and an antibiotic, or any combination thereof.

In an embodiment of the present disclosure, the antibiotic is fluoroquinolone, optionally along with antibiotic selected from group comprising lincosamide, macrolide, retinoid, tetracycline and benzoyl peroxide, or any combination thereof.

In another embodiment of the present disclosure, the fluoroquinolone antibiotic is selected from group comprising besifloxacin, nadifloxacin and levafloxacin, or combinations thereof; wherein the lincosamide is clindamycin; wherein the macrolide is erythromycin; and wherein the retinoid is adapalene.

In yet another embodiment of the present disclosure, the kit is used for detecting mutation in 23S rRNA of P. acnes; wherein the mutation is selected from A2058G (E. coli numbering), A2059G (E. coli numbering) or a combination thereof with respect to wild type P. acnes; wherein the restriction endonuclease is BpuJI; and wherein the DNA endonuclease is a mismatch specific DNA endonuclease selected from group comprising CELII, CELI and T7 endonuclease.

In yet another embodiment of the present disclosure, the kit is used for detecting mutation in 23S rRNA of P. acnes; wherein the mutation is A2058G (E. coli numbering) with respect to wild type P. acnes.

In yet another embodiment of the present disclosure, when the mutation detected is A2058G or a combination of A2058G and A2059G, the P. acnes detected is rendered resistant to antibiotic selected from lincosamide and macrolide or a combination thereof.

In yet another embodiment of the present disclosure, when the mutation detected is A2058G or a combination of A2058G and A2059G, the P. acnes detected is rendered resistant to antibiotic selected from clindamycin, erythromycin or a combination thereof.

In yet another embodiment of the present disclosure, the antibiotic is present at concentration ranging from about 0.1% w/w to about 10% w/w; and wherein the antibiotic in form of a formulation selected from group comprising topical formulation, solid oral formulation, liquid oral formulation, inhalation formulation, nasal formulation, ophthalmic formulation, parenteral formulation, phytoceutical, nutraceutical and food stuff or any combination thereof.

In yet another embodiment of the present disclosure, the antibiotic B is present at concentration ranging from about 0.2% w/w to about 5% w/w, in a further embodiment, the antibiotic B is present at concentration ranging from about 1% w/w to about 2% w/w.

In yet another embodiment of the present disclosure, the antibiotic B is in form of a topical formulation.

In still another embodiment of the present disclosure, the topical formulation is selected from group comprising cream., gel, spray, foam, lotion or any combination thereof; the solid oral formulation is selected from group comprising tablet, capsule, troche, lozenge, dispersible powder, dispersible granule or any combination thereof; the liquid oral formulation is selected from group comprising aqueous or oily suspension, emulsion, drop, emulsion in hard or soft gel capsule, syrup, elixir or any combination thereof; the parenteral formulation is selected from group comprising intravenous injection, intramuscular injection, intramuscular depot, subcutaneous injection, percutaneous injection or any combination thereof; the inhalation formulation is selected from group comprising inhaler, dry powder inhaler, nebulizer or any combination thereof; the nasal formulation is selected from group comprising nasal drops, nasal sprays or a combination thereof; and the ophthalmic formulation comprises eye drops.

Precision medicine is the future, and the treatment of acne needs to evolve in that direction. A key step towards rationalizing the use of antibiotics is to ensure that patients are treated with the most appropriate antibiotic for quick relief as well as to have a check on the rising of emergence of resistance. The existing prescriptions to curtail the resistance issue are inadequate to stave off the impact of acne vulgaris as well as due to the growing number of bacteria/strains exhibiting resistance in acne therapy.

Further, to overcome the deficiencies in the prior art, there is a need to develop inventions that will enable rapid detection of P. acnes specific region within the conserved 23S rRNA domain, to enable easy and quick identification of P. acnes present among other flora in samples and also confirm antibiotic resistant mutations in the 23S rRNA of P. acnes.

In this regard, the present disclosure relates to a method of detecting P. acnes in a sample, wherein the sample is contacted with a pair of oligonucleotide primers set forth in SEQ ID No. 1 and SEQ ID No. 2. and subjecting to polymerase chain reaction (PCR) amplification. Here, SEQ ID No. 1 is the forward primer, while SEQ ID No. 2 is the reverse primer.

Forward Primer-
SEQ ID No. 1
5′-CTGTGAGTGTGATGCGTAGC-3′
Reverse Primer-
SEQ ID No. 2
5′-ACATCGAGGTGCCAAACCAT-3′

The present disclosure also relates to a method of detecting P. acnes in a sample, wherein the sample is contacted with a pair of oligonucleotide primers complementary to the primer sequences disclosed above and as set forth in SEQ ID No. 7 and SEQ ID No. 8 and subjecting to polymerase chain reaction (PCR) amplification. Here, SEQ ID No. 7 is the forward primer, while SEQ ID No. 8 is the reverse primer.

Forward Primer-
SEQ ID No. 7
5′-GACACTCACACTACGCATCG-3′
Reverse Primer-
SEQ ID No. 8
5′-TGTAGCTCCACGGTTTGGTA-3′

The inventors of the present disclosure have recognized a particular region containing the nucleotide that when mutated confers clindamycin resistance within the 23S rRNA of P. acnes which is highly specific for P. acnes, and accordingly designed primers towards this specific region such that they would only amplify this P. acnes specific region and not the 23S rRNA domain of any other microorganisms. Thus, upon subjecting a sample to PCR amplification with the primers of SEQ ID Nos. 1 and 2 or with the primers of SEQ ID Nos. 7 and 8 of the present disclosure, if an amplified product is obtained, the presence of P. acnes in the sample is confirmed, thus enabling its detection.

Additionally, since the primers of the present disclosure are highly specific, the sample to be detected is directly contacted with the primers for detecting P. acnes, without the requirement of culturing and isolating P. acnes. Particularly, the sample is inoculated in PCR tube comprising amplification reagents such as dNTPs, magnesium chloride, buffer, nuclease free water and DNA polymerase along with the primers of SEQ ID Nos. 1 and 2 or the primers of SEQ ID Nos. 7 and 8 and subjected to PCR During the initial denaturation step, the P. acnes cells in the sample get lysed and the genomic DNA is then amplified.

In an embodiment, the PCR amplification comprises initial denaturation at temperature ranging from about 90° C. to about 97° C. for about 5-10 minutes, preferably at temperature of about 94° C. for about 10 minutes. This is followed by 30-40 cycles of denaturation at temperature ranging from about 90° C. to about 97° C. for about 30-90 seconds, preferably at temperature of about 94° C. for about 45 seconds. This is followed by annealing at temperature ranging from about 48° C. to about 52° C. for about 0.5-2 minutes, preferably at temperature of about 50° C. for about 1 minute. This is then followed by elongation at temperature of about 68° C. to about 72° C. for about 0.5-2 minutes, preferably at temperature of about 72° C. for about 1 minute. This is finally followed by a final elongation at temperature ranging from about 68° C. to about 72° C. for about 5-10 minutes, preferably at temperature of about 72° C. for about 5 minutes.

In another embodiment of the present disclosure, the region of the genomic sequence of 23S rRNA recognized by the primers of SEQ ID Nos. 1 and 2 also contain positions 2058 and 2059 (as per E. coli numbering). Thus, the amplified product obtained using said primers would also contain the above-mentioned positions, and any mutations in said positions, such as A2058G (as per E. coli numbering) and A2059G (as per E. coli numbering) would also be present in the amplified products.

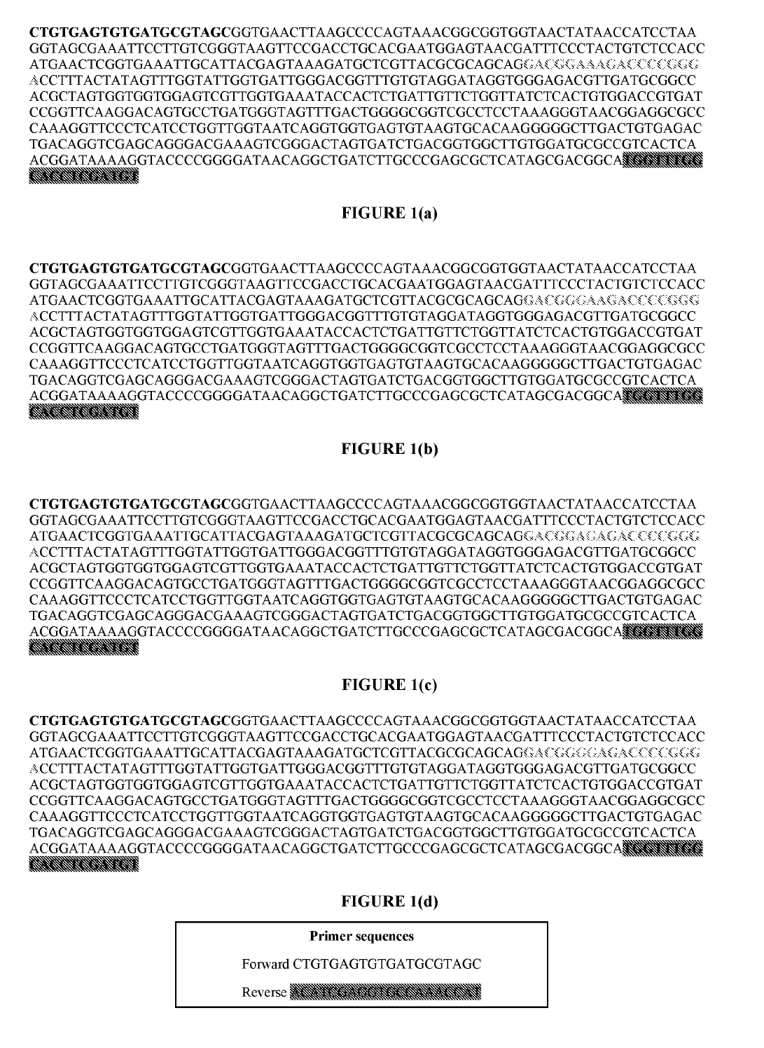

In another embodiment, the primers of SEQ ID Nos. 1 and 2 and the primers of SEQ Nos. 7 and 8 amplify DNA encoding 23S rRNA sequence of P. acnes (wild type)-SEQ ID No. 3 (FIG. 1(a)), and also amplify DNA encoding 23S rRNA sequence of P. acnes having mutation(s) (SEQ ID Nos. 4, 5 or 6) (FIGS. 1(b)-1(d). Thus, there is no requirement of separate sets of oligomers for identifying the presence of P. acnes and for identifying/amplifying the region of mutation.

The DNA sequence encoding the 23S rRNA sequence of a wild type P. acnes strain (SEQ ID No. 3), DNA sequence encoding the 23S rRNA sequence of P. acnes strain carrying the A2058G mutation (SEQ ID No. 4), DNA sequence encoding the 23S rRNA sequence of P. acnes strain carrying the A2059G mutation (SEQ ID No. 5) and DNA sequence encoding the 23S rRNA sequence of P. acnes strain carrying the A2058G and the A2059G mutation (SEQ ID No. 6) are depicted in FIGS. 1(a), 1(b), 1(c) and 1(d) respectively. As can be observed from the sequences provided, the 23S rRNA sequence of the mutant strain of FIG. 1(b) contains the nucleotide ‘G’ in position 2058 instead of the nucleotide ‘A’ as seen in the wild type strain, the 23S rRNA sequence of the mutant strain of FIG. 1(c) contains the nucleotide ‘G’ in position 2059 instead of the nucleotide ‘A’ as seen in the wild type strain and the 23S rRNA sequence of the mutant strain of FIG. 1(d) contains the nucleotide ‘G’ in positions 2058 and 2059 instead of the nucleotide ‘A’ as seen in the wild type strain. FIG. 1 also shows the sequences of the forward and reverse primers (SEQ ID Nos. 1 and 2) of the present disclosure. Further, the specific regions of the DNA encoding 23S rRNA sequence to which the primers of the instant disclosure are synthesized are depicted in FIG. 1.

The present disclosure also relates to a method for detecting presence of a single point mutation A2058G in the 23S rRNA sub-unit of P. acnes. The method is based on the amplification of a region on the 23S rRNA genomic sequence that is specific for P. acnes and containing the clindamycin resistance conferring region followed by detection of the mutation through selective action of an endonuclease at a mismatch at the site of said mutation.

In an embodiment, the method involves a two-step assay, wherein DNA encoding the 23S rRNA sequence of P. acnes is subjected to PCR amplification, following which the presence of the single point mutation is determined. The presence of the single point mutation is determined using restriction endonuclease, or DNA hybridization followed by endonuclease activity. The signature of bands obtained by agarose gel pattern after cleavage with a particular endonuclease selectively screens the A2058G clindamycin resistant P. acnes.

In an embodiment, the method of the present disclosure is quick and cost-effective since there is no requirement for culturing and isolating P. acnes from the sample prior to the amplification and detection steps. This is because, as disclosed earlier, the primers used in the first step of the method of present disclosure (SEQ ID Nos. 1 & 2/SEQ ID Nos. 7 and 8) are designed in such a way that they recognize and amplify a particular region of P. acnes 23S rRNA genomic sequence that is unique for the particular species (not present in any other bacteria or any other Propionibacterial species other than P. acnes) and which also contains the crucial 2058 nucleotide, conferring clindamycin-macrolide resistance. Thus, the primers of the present disclosure selectively amplify the P. acnes specific 23S rRNA genomic sequence, using PCR (polymerase chain reaction) directly from samples, for examples, samples collected from the acne lesions of subjects. This also ensures that there are no false positive results caused by amplification of 23S rRNA genomic sequence of other bacterial species present in the samples.

In an embodiment, the sample may be selected from group comprising water, food, environmental sample and biological sample. In another embodiment, the sample is a biological sample selected from group comprising tissue, skin, acne lesion, bone and body fluids including but not limited to blood.

In an embodiment of the present disclosure, for PCR amplification to occur, the sample comprising P. acnes or the DNA sequence encoding 23S rRNA of P. acnes is contacted with the forward and reverse primers of SEQ ID Nos. 1 and 2 or with the forward and reverse primers of SEQ ID Nos. 7 and 8 and subjected to amplification in the presence of amplification reagents under the same conditions, to obtain amplified product of 646 bp. In a further embodiment, the amplification reagents are selected from a group comprising magnesium chloride, dNTPs, DNA polymerase, nuclease free water and buffer.

In an embodiment, the amplified product obtained is analyzed to detect the presence of single point mutation, at position 2058 of the 23S rRNA sequence, i.e., A2058G. Such detection is carried out either by restriction digestion, or by DNA based hybridization, followed by endonuclease digestion.

In an embodiment, for detecting the A2058G point mutation, the 646 bp amplified product is digested with restriction endonuclease, BpuJI. If the mutation is present, BpuJI cleaves the sequence into two portions of 198 bp and 447 bp. However, if there is no mutation, BpuJI does not recognize the sequence and thus no cleaving of the amplified product takes places. The cleaved/uncleaved products are visualized using agarose gel electrophoresis, wherein the presence of two bands confirms mutation and presence of one band confirms absence of mutation.

In another embodiment, for detecting the A2058G point mutation, the 646 bp amplified product is contacted with 23S rRNA amplified product of wild type P. acnes and subjected to hybridization by melting/denaturing the nucleic acids at temperature ranging from about 90° ^{C.} to about 95° C. for time period ranging from about 5-10 minutes, preferably at temperature of about 95° C. for about 10 minutes. This is followed by reannealing of the strands by gradually decreasing the temperature in step down manner to about 20° C.-27° C., preferably to a temperature of about 25° C. Upon reannealing, if a homoduplex is obtained, the 23S rRNA genomic sequence of P. acnes does not contain any mutation. On the other hand, if a heteroduplex is formed, the P. acnes 23S rRNA genomic sequence contains the A2058G mutation. Further, the presence of homoduplex or heteroduplex is detected by a mismatch specific DNA endonuclease. If after digestion with the endonuclease, visualization of the product using agarose gel electrophoresis shows the presence of additional low molecular weight band(s), it indicates the presence of heteroduplex DNA (nicked by endonuclease), thereby confirming the presence of A2058G mutation. On the other hand, presence of a single band indicates the presence of homoduplex DNA, thereby confirming the absence of A2058G mutation, since there will be no endonuclease action on the homoduplex DNA.

In an alternate embodiment, the point mutation is detected by carrying out two DNA hybridizations parallelly: One of the 646 bp amplified product with 23S rRNA PCR product of wild type P. acnes and the other of the 646 bp amplified product with 23S rRNA PCR product of P. acnes containing A2058G. If a heteroduplex is formed in the first case and a homoduplex is formed in the second case, the P. acnes 23S rRNA genomic sequence being detected contains the A2058G mutation. The presence of heteroduplex and homoduplex is confirmed after DNA endonuclease treatment, by the presence of additional low molecular weight band(s) in the first case and the presence of one band in the second case, after agarose gel electrophoresis. In another alternate embodiment, detection of the 2058G mutation is carried out by hybridization of the 646 bp amplified product with 23S rRNA amplified product of P. acnes containing A2058G, followed by endonuclease treatment. Presence of a single band upon running on Agarose gel electrophoresis confirms presence of the A2058G mutation. However, presence of additional low molecular weight band(s) upon running on agarose gel electrophoresis confirms absence of A2058G mutation.

In an embodiment, the DNA endonuclease is selected from group comprising CELII, CELI and T7 endonuclease.

The present disclosure further relates to a method for detecting any point mutation in 23S rRNA of P. acnes or a combination of point mutation A2058G with other mutations in 23S rRNA of P. acnes. The method involves contacting a sample comprising P. acnes or contacting the DNA sequence encoding 23S rRNA of P. acnes with the forward and reverse primers of SEQ ID Nos. 1 and 2 or with the forward and reverse primers of SEQ ID Nos. 7 and 8 and subjecting to amplification as detailed above. Post-amplification, based on the specific mutation(s) to be detected, the amplified product is subjected to different restriction endonuclease(s) which will cleave the amplified product at the point of mutation(s).

In an embodiment, the point mutation(s) may also be detected by DNA hybridization of the 646 by amplified product with 23S rRNA PCR product of wild type P. acnes and detecting the presence/absence of the mutation(s) by means of a mismatch specific DNA endonuclease as disclosed above. For instance, if multiple mutations are present on the amplified product, there will be mis-matches at multiple places in the heteroduplex, which will be cleaved by the endonuclease resulting in multiple bands upon visualization using agarose gel electrophoresis.

In an alternate embodiment, the point mutation(s) is detected by DNA hybridization of the 646 bp amplified product with 23S rRNA amplified product of wild type P. acnes and parallelly DNA hybridization of the 646 bp amplified product with 23S rRNA amplified product of P. acnes containing A2058G. The formation of heteroduplex in both cases confirms the presence of mutation at a site other than 2058 residue, which is confirmed by the presence of additional low molecular weight band(s) upon running in agarose gel electrophoresis after DNA endonuclease treatment. In another alternate embodiment, detection of mutation(s) is carried out by hybridization of the 646 bp amplified product with 23S rRNA amplified product of P. acnes containing A2058G, followed by endonuclease treatment. Presence of additional low molecular weight band(s) upon running on agarose gel electrophoresis confirms presence of the mutation at site other than 2058 residue.

In another embodiment, point mutation at the 2059 residue of 23S rRNA of P. acnes is detected by hybridization of the 646 bp amplified product with 23S rRNA amplified product of wild type P. acnes and parallelly DNA hybridization of the 646 bp amplified product with 23S rRNA amplified product of P. acnes containing A2059G. Upon running on agarose gel electrophoresis after DNA endonuclease treatment, the presence of additional low molecular weight band(s) in the first case and the presence of one band in the second case confirms that the genomic sequence of the 23S rRNA of the P. acnes being detected contains the A2059G mutation. In an alternate embodiment, detection of the A2059G mutation is carried out by hybridization of the 646 by amplified product with 23S rRNA amplified product of P. acnes containing A2059G, followed by endonuclease treatment. Presence of a single band upon running on agarose gel electrophoresis confirms presence of the mutation, while presence of additional low molecular weight band(s) confirms absence of A2059G mutation.

In a further embodiment, a combination of mutations at the 2058 and 2059 residues of 23S rRNA of P. acnes is detected by hybridization of the 646 bp amplified product with 23S rRNA amplified product of wild type P. acnes and parallelly DNA hybridization of the 646 bp amplified product with 23S rRNA amplified product of P. acnes containing A2058G and A2059G. Upon running on agarose gel electrophoresis after DNA endonuclease treatment, the presence of additional low molecular weight band(s) in the first case and the presence of one band in the second case confirms that the genomic sequence of the 23S rRNA of the P. acnes being detected contains the A2058G and the A2059G mutations. Alternately, the combination of mutations at the 2058 and 2059 residues is detected by hybridization of the 646 bp amplified product with 23S rRNA amplified product of P. acnes containing A2058G- and A20596, followed by endonuclease treatment. Presence of a single band upon running on agarose gel electrophoresis confirms presence of the mutations, while presence of additional low molecular weight band(s) confirms absence of the mutations.

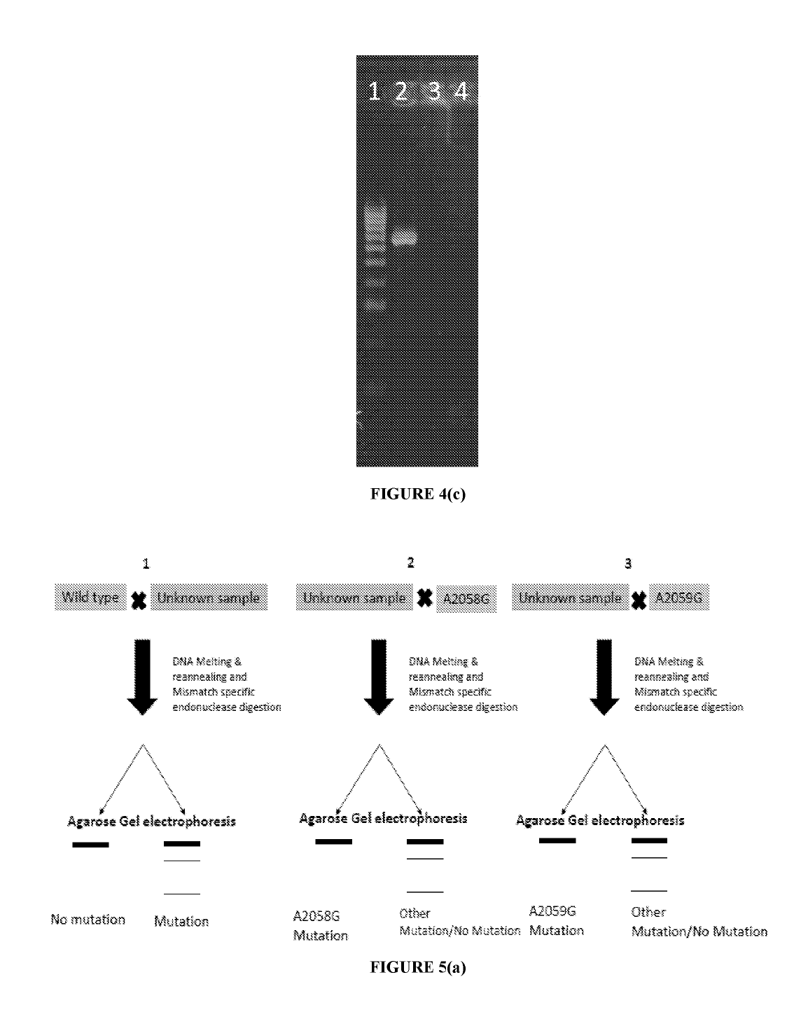

The different possible hybridizations followed by endonuclease digestion for detecting mutation(s) in the genomic sequence of P. acnes as detailed above are depicted in FIG. 5(a).

The present disclosure also relates to identification of antibiotic resistance in P. acnes present in a sample. The identification of antibiotic resistance is carried out by detecting point mutation in the 23S rRNA sequence of P. acnes by the methods of the present disclosure. Presence of specific point mutations confers antibiotic resistance in P. acnes, thus enabling the identification.

Table 1 below depicts the antimicrobial susceptibility patterns of P. acnes isolates against different antibiotics:

TABLE 1
Antimicrobial Susceptibility Patterns of P. acnes Isolates Against
Different Antibiotics

| | P. acnes | | Mutation | | | | |
|---|---|---|---|---|---|---|---|
| S. | Isolate | | (23S rRNA) | | MIC (μg/ml) | | |
| No. | No. | Phylotype | Type | Position | Clindamycin | Erythromycin | Fluoroquinolone |
| 1 | V21A2 | IB | I | A2058G | 8-64 | 50-200 | 0.25 |
| 2 | V21A3 | IB | I | A2058G | 8-32 | 100-200 | 0.25 |
| 3 | V21A4 | IB | I | A2058G | 16-64 | 100-200 | 0.25 |
| 4 | V21A9 | IB | I | A2058G | 16-32 | 100-200 | 0.25 |
| 5 | V21A10 | IB | I | A2058G | 16-64 | 100-200 | 0.25 |
| 6 | V21B2 | IB | I | A2058G | 16-64 | >200 | 0.25 |
| 7 | V21B3 | IB | I | A2058G | 64 | >200 | 0.25 |
| 8 | V21B4 | IB | I | A2058G | 32-64 | >200 | 0.25 |
| 9 | V21B6 | IB | I | A2058G | 16-64 | >200 | 0.5 |
| 10 | V21B7 | IB | I | A2058G | 16-64 | >200 | 0.5 |
| 11 | V21B8 | IB | I | A2058G | 16-64 | >200 | 0.5 |
| 12 | V21A1 | IA1 | IV | A2059G | 1-8 | 50-200 | 0.25 |
| 13 | V21A5 | IA1 | IV | A2059G | 1 | 100-200 | 0.25 |
| 14 | V21A6 | IA1 | IV | A2059G | 1-2 | 100-200 | 0.5 |
| 15 | V21A7 | IA1 | IV | A2059G | 0.5-2 | 100-200 | 0.25 |
| 16 | V21A8 | IA1 | IV | A2059G | 1-2 | 100-200 | 0.25 |

As can be observed from Table 1, P. acnes strains having the A2059G mutation (shaded) are less resistant to clindamycin compared to strains having the A2058G mutation.

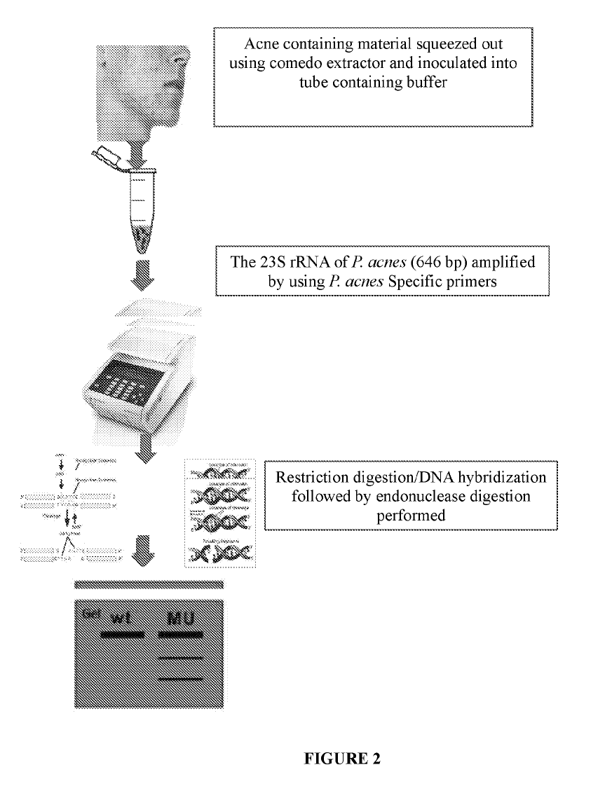

In a non-limiting embodiment, identification of antibiotic resistance of P. acnes is carried out as depicted in FIG. 2, wherein acne containing material is squeezed out using extracter and inoculated into a tube containing buffer. The DNA encoding the 23S rRNA of P. acnes is then subjected to PCR amplification using primers of SEQ ID Nos. to 1 and 2 or primers of SEQ ID Nos, 7 and 8, following which point mutation(s) is detected by performing restriction digestion or DNA hybridization by the methods detailed above. If the genomic sequence of 23S rRNA of P. acnes from the sample is found to contain A2058G mutation, or a combination of A2058G mutation with other mutations, the P. acnes is identified as lincosamide resistant, particularly, clindamycin resistant and the patient from whom the sample is obtained is diagnosed as being infected with lincosamide resistant, particularly, clindamycin resistant P. acnes. Further, mutation at A2058 disrupts the pattern of hydrogen bonding with erythromycin, therefore impairing binding and rendering bacteria resistant. Thus, this approach also detects macrolide (erythromycin) resistance in P. acnes.

In an embodiment, if the 23S rRNA of P. acnes from the sample does not carry A2058G mutation or any other mutation, the P. acnes is identified as lincosamide and macrolide sensitive, particularly, clindamycin and erythromycin sensitive and the patient from whom the sample is obtained is diagnosed as being infected with lincosamide and macrolide sensitive, particularly, clindamycin and erythromycin sensitive P. acnes.

In another embodiment, if the 23S tRNA of P. acnes from the sample carries only A2059G mutation, the P. acnes is identified as macrolide resistant, but less resistant to lincosamide, particularly less resistant to clindamycin, compared to strains having A2058G mutation.

In another embodiment, the sample may be any sample containing P. acnes and may be selected from group comprising water, food, environmental and biological samples. In another embodiment, the sample is a biological sample selected from group comprising tissue, skin, acne lesion, bone and body fluids including but not limited to blood.

The present disclosure further relates to method of determining an antibiotic regimen for a patient infected with P. acnes. The method involves identifying/diagnosing the resistance of P. acnes from a sample of the patient to an antibiotic A by the methods disclosed above and administering the patient with an antibiotic B if the P. acnes is resistant to antibiotic A.

In an embodiment, the antibiotic B is any antibiotic capable of treating the resistant P. acnes strains.

In a non-limiting embodiment, if the P. acnes from the sample of the patient is found to contain A2058G mutation in 23S rRNA sequence, the patient is diagnosed as being infected with lincosamide and/or macrolide resistant P. acnes and accordingly the patient is administered an antibiotic other than lincosamide and macrolides with or without retinoids and/or benzoyl peroxide (BPO).

In a particular non-limiting embodiment, the patient is administered a fluoroquinoline-based antibiotic optionally along with adapalene, BPO, tetracycline class of antibiotics or combinations thereof.

In another non-limiting embodiment, the fluoroquinolone-based antibiotic is selected from group comprising nadifloxacin, besifloxacin, and levofloxacin.

In another embodiment of the present disclosure, if the P. acnes from the sample of the patient is not found to contain any mutation, the patient is diagnosed as being infected with lincosamide and macrolide sensitive P. acnes and accordingly the patient is administered a lincosamide or macrolide antibiotic optionally along with BPO, retinoids such as adapalene, or combinations thereof.

In another embodiment of the present disclosure, if the P. acnes from the sample of the patient is found to contain A2059G mutation in 23S rRNA sequence, the patient is administered an antibiotic selected from lincosamide or fluoroquinolone optionally along with BPO, retinoids such as adapalene, or combinations thereof.

In a non-limiting embodiment, the fluoroquinolone-based antibiotic is selected from group comprising nadifloxacin, besifloxacin, and levofloxacin and the lincosamide antibiotic is clindamycin.

In yet another embodiment of the present disclosure, the antibiotics are administered as a topical formulation, solid oral formulation, liquid oral formulation, inhalation formulation, nasal formulation, ophthalmic formulation, parenteral formulation, phytoceutical, nutraceutical and food stuff or any combination thereof. In a preferred embodiment, the antibiotics are administered as a topical formulation.

In yet another embodiment of the present disclosure, the topical formulation is selected from group comprising cream, gel, spray, foam, lotion or any combination thereof; the solid oral formulation is selected from group comprising tablet, capsule, troche, lozenge, dispersible powder, dispersible granule or any combination thereof; the liquid oral formulation is selected from group comprising aqueous or oily suspension, emulsion, drop, emulsion in hard or soft gel capsule, syrup, elixir or any combination thereof; the parenteral formulation is selected from group comprising intravenous injection, intramuscular injection, intramuscular depot, subcutaneous injection, percutaneous injection or any combination thereof; the inhalation formulation is selected from group comprising inhaler, dry powder inhaler, nebulizer or any combination thereof; the nasal formulation is selected from group comprising nasal drops, nasal sprays or a combination thereof; and the ophthalmic formulation comprises eye drops.

In yet another embodiment of the present disclosure, the antibiotics are combined with pharmaceutically acceptable excipients for preparing formulations. The pharmaceutically acceptable excipient is selected from group comprising gum, granulating agent, binder, lubricant, disintegrating agent, sweetening agent, additive, solvent, glidant, anti-adherent, anti-static agent, anti-oxidant, surfactant, viscosity enhancer, plant cellulosic material, coloring agent, flavoring agent, coating agent, plasticizer, preservative, suspending agent, emulsifying agent and spheronization agent or any combinations thereof.

In an embodiment, the antibiotic in the formulation is present at concentration ranging from about 0.1% w/w to about 10% w/w, preferably from about 0.2% w/w to about 5% w/w and more preferably from about 1% w/w to about 2% w/w.

In yet another embodiment of the present disclosure, the topical formulation of antibiotics is applied at least once daily but not more than twice daily.

In yet another em.bodiment of the present disclosure, the oral formulation of antibiotics is administered at least once daily and up to four times daily.

The present disclosure further relates to a kit for detecting P. acnes or for detecting mutation in 23S rRNA sequence of P. acnes comprising primer sequences of SEQ ID Nos. 1 and 2 or primer sequences of SEQ ID Nos. 7 and 8, or both, optionally along with dNTPs, DNA polymerase, buffer, nuclease free water, magnesium chloride, vials, restriction endonuclease BpuJI, nucleotide mismatch specific DNA endonuclease selected from group comprising CELII, CELI and T7 endonuclease, P. acnes wild type strain such as P. acnes MTCC 1951 and P. acnes MTCC 3297, DNA encoding 23S rRNA of P. acnes containing A2058G mutation, DNA encoding 23S rRNA of P. acnes containing A2059G mutation, DNA encoding 23S rRNA of P. acnes containing A2058G and A2059G mutation, instruction manual, or any combination thereof.

The present disclosure further relates to kits for detecting antibiotic resistance in P. acnes and treating infections caused by P. acnes, wherein apart from the components mentioned in the kit above, the kit would also include antibiotics.

In an embodiment, the kit would include fluoroquinolone-based antibiotics optionally along with BPO, retinoids such as adapalene, tetracycline class of antibiotics or combinations thereof. The kit could also include lincosamide and macrolide class of antibiotics such as clindamycin and erythromycin, optionally along with BPO, retinoids such as adapalene, tetracycline class of antibiotics or combinations thereof.

In another embodiment, the fluoroquinolone-based antibiotic is selected from group comprising nadifloxacin, besifloxacin, and levofloxacin.

In another embodiment of the present disclosure, the antibiotic(s) in the form of a formulation selected from group comprising topical formulation, solid oral formulation, liquid oral formulation, inhalation formulation, nasal formulation, ophthalmic formulation, parenteral formulation, phytoceutical, nutraceutical and food stuff or any combination thereof, preferably as a topical formulation.

In yet another embodiment of the present disclosure, the topical formulation is selected from group comprising cream, gel, spray, foam, lotion or any combination thereof; the solid oral formulation is selected from group comprising tablet, capsule, troche, lozenge, dispersible powder, dispersible granule or any combination thereof; the liquid oral formulation is selected from group comprising aqueous or oily suspension, emulsion, drop, emulsion in hard or soft gel capsule, syrup, elixir or any combination thereof; the parenteral formulation is selected from group comprising intravenous injection, intramuscular injection, intramuscular depot, subcutaneous injection, percutaneous injection or any combination thereof; the inhalation formulation is selected from group comprising inhaler, dry powder inhaler, nebulizer or any combination thereof, the nasal formulation is selected from group comprising nasal drops, nasal sprays or a combination thereof; and the ophthalmic formulation comprises eye drops.

In yet another embodiment of the present disclosure, the antibiotic formulation in the kit comprises antibiotic in combination with pharmaceutically acceptable excipients selected from group comprising gum, granulating agent, binder, lubricant, disintegrating agent, sweetening agent, additive, solvent, glidant, anti-adherent, anti-static agent, anti-oxidant, surfactant, viscosity enhancer, plant cellulosic material, coloring agent, flavoring agent, coating agent, plasticizer, preservative, suspending agent, emulsifying agent and spheronization agent or any combinations thereof.

In an embodiment, the antibiotic in the formulation is present at concentration ranging from about 0.1% w/w to about 10% w/w, preferably from about 0.2% w/w to about 5% w/w and more preferably from about 1% w/w to about 2% w/w.

Additional embodiments and features of the present disclosure will be apparent to one of ordinary skill in the art based upon the description provided herein. The embodiments herein provide various features and advantageous details thereof in the description. Descriptions of well-known/conventional methods and techniques are omitted so as to not unnecessarily obscure the embodiments herein. Further, the invention herein provides for examples illustrating the above described embodiments, and in order to illustrate the embodiments of the present invention, certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1

### Designing Specific Primers for Amplifying a Region of 23S rRNA Sequence that is Specific for P. acnes and Determining the Specificity of the Primers

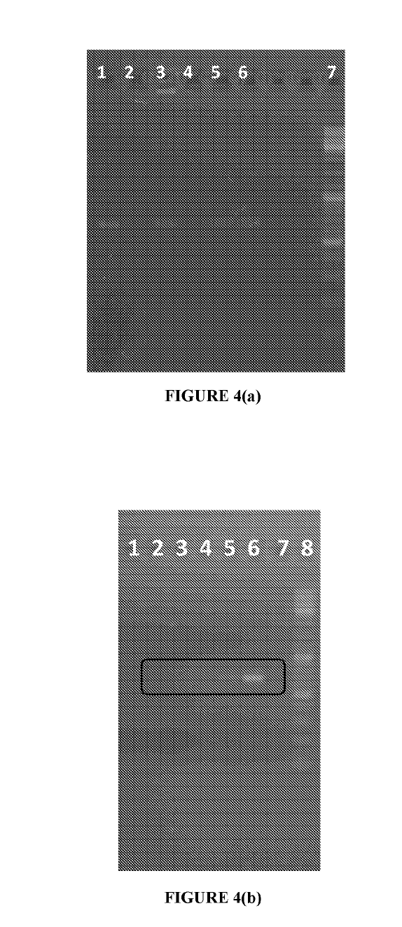

Primers (Forward 5′-CTGTGAGTGTGATGCGTAGC-3′ and Reverse 5′-ACATCGAGGTGCCAAACCAT-3′) are designed to specifically amplify a region of 23S rRNA genomic sequence that is specific for P. acnes. The primers thus designed are used for PCR amplification of the DNA encoding for the 23S tRNA sequence of the following P. acnes strains: MTCC 1951 and MTCC 3297 (standard wild type strains), CCARM 9010 and V21B2 (clindamycin resistant strains), and V21A6 and V21A7 (containing A2059G mutation). The PCR reaction is setup for initial denaturation at about 94° C. for about 10 minutes followed by 30 cycles of denaturation at temperature of about 94° C. for about 45 seconds, annealing at temperature of about 50° C. for about 1 minute, elongation at temperature of about 72° C. for about 1 minute and by final elongation at temperature of about 72° C. for about 5 minutes. About 5 μl of the product is run in 1.5% agarose gel electrophoresis. It is observed that amplified PCR products of 646 bp are obtained from all the P. acnes strains as observed by agarose gel electrophoresis (FIG. 4(a)).

To further confirm the specificity of the primers towards P. acnes, the above-mentioned primers are used to amplify the 23S region of Staphylococcus epidermidis (M3-1 & M4-1) and P. avidum (S15-A1 & S3-1) using the same PCR reaction conditions and 10 μL of the product is run in 1.5% agarose gel electrophoresis. As depicted in FIG. 4(b), no PCR product is obtained for the 23S region of Staphylococcus epidermidis and. P. avidum (Lanes 1-4 show no bands), while PCR product is obtained for the P. acnes wild type and mutated 23S rRNA (Lanes 5 and 6 show bands). This indicates that the primer sequences of the present disclosure are highly specific to P. acnes species.

As a further confirmatory experiment to test the specificity of the primers, two mixed cultures are obtained and both samples are separately contacted with the primers of the present disclosure and subjected to PCR under the same reaction conditions. About 5 μl of the product from each sample is run in 1.5% agarose gel electrophoresis. As can be seen in FIG. 4(c), only one band (Lane 2) of 646 bp is obtained, while Lane 3 shows no band. Further, identification of the microorganisms present in each sample shows that Mixture culture 1 (Lane 2) contains S. epidermidis, S. aureus, P. avidum and P. acnes while Mixture culture 2 (Lane 3) contains S. epidermidis, S. aureus and P. avidum. Thus, it is confirmed that the primers of the present disclosure selectively detect P. acnes and no other microorganisms since an amplified product is only obtained with respect to the sample containing P. acnes.

### Example 2

### Detection of A2058G Mutation in P. acnes 23S rRNA Sequences using BpuJI Restriction Endonuclease

Restriction enzymes/restriction endonucleases can recognize and cleave DNA in specific nucleotide sequences. To detect the 2058 mutation in P. acnes 23S rRNA sequence, suitable restriction enzymes are screened using NEB cutter website. By using this tool four restriction enzymes (BpuJI, PsuGI, Sth1321, AspBHI) are shortlisted based on their affinity to recognize the A2058G mutation. However, apart from BpuJI, other enzymes show multiple cleavable sites in 23S rRNA sequences. BpuJI specifically recognizes the sequence 5′-CCCGT-3′ which falls within nucleotide A2058G (E. coli equal numbering) mutated sequences in P. acnes 23S rRNA sequence.

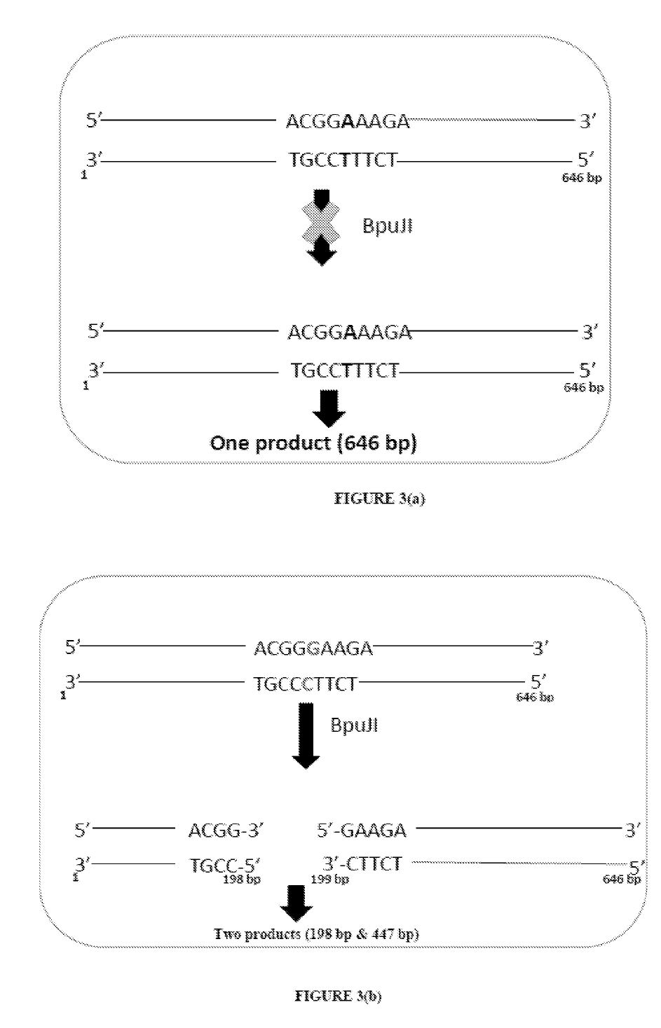

A 646 bp amplified sequence of DNA encoding 23S rRNA is obtained with respect to the wild type P. acnes strain using primers of SEQ ID Nos. 1 and 2, under the same conditions as provided in Example 1. This sequence and the 646 bp amplified sequence of mutated (A2058G) P. acnes strain obtained in Example 1 are subjected to restriction digestion using BpuJI. As shown in FIG. 3(a), when the wild type sequence is subjected to the Enzyme BpuJI, BpuJI does not recognize the wild type sequence and hence does not cleave the DNA and a single product of 646 bp is obtained. On the other hand, as shown in. FIG. 3(b), when 23S rRNA amplified sequence of the P. acnes with A2058G mutation is subjected to the Enzyme BpuJI, BpuJI recognizes the sequence containing mutation A2058G and cleaves the DNA at 198^{th} bp, leading to two products of 198 bp and 447 bp. This confirms that restriction enzyme can be used for detecting A2058G mutation in P. acnes 23S rRNA sequence.

### Example 3

### Detection of A2058G and Other Point Mutations in Genomic DNA of Clindamycin Resistance Conferring 23S rRNA using DNA Hybridization

In a PCR tube, equal volume (about 10 μl) of the 23S rRNA PCR product of wild type P. acnes (MTCC 1951) and the 23S rRNA PCR product from a clindamycin resistant P. acnes (CCARM 9010) obtained in Example 1 is added and subjected to melting by heating the tube at about 95° C. for about 10 minutes. This is followed by reannealing of the strands by gradually decreasing the temperature in step down manner to about 25° C., The reannealed sample containing the heteroduplex DNA is digested with a mismatch specific DNA endonuclease (Surveyor®). The mismatch specific DNA endonuclease recognizes the base substitution mismatch site at the 2058 nucleotide of 646 bp PCR product and cleaves the DNA. Samples are stored at about −20° C. and whole samples (about 20 μl) are loaded in 2.0% agarose gels, wherein cleaving of the DNA is confirmed by an additional band at 450 bp (FIG. 5(b)-Lanes 2 and 3) over and above the undigested band at 646 bp. This additional band is not observed when the same process is repeated with 23S rRNA PCR product of clindamycin sensitive P. acnes (MTCC 1951), i.e., hybridization of MTCC 1951+MTCC 1951 (about 10 μl each) (FIG. 5(b)-Lane 4). This shows that when the wild type strains are hybridized, they form a homoduplex and thus the DNA endonuclease does not cleave the sequence as a result of which only one band is observed.

Thus, DNA hybridization techniques helps in detecting mutation, particularly, A2058G mutation in 23S rRNA sequence of P. acnes.

### Example 4

### Activity of Fluoroquinolone Antibiotics on Clindamycin Resistant P. acnes

Various clinical isolates are obtained from patients carrying Clindamycin Resistant P. acnes and their antimicrobial susceptibility to fluoroquinolone antibiotics is studied vis-à-vis susceptibility to Clindamycin and Erythromycin. The results are tabulated in Table 2 below:

TABLE 2
P. acnes clinical isolates (clindamycin resistant) antimicrobial
susceptibility against various antibiotics

| | MIC (μg/ml) | | | | |
|---|---|---|---|---|---|
| P. acnes | Clinda- | Erythro- | Levo- | Nadi- | Besi- |
| strain No. | mycin | mycin | floxacin | floxacin | floxacin |
| V21A-1 | 1-8 | 50-200 | <0.13 | <0.13 | 0.25 |
| V21A-2 | 8-64 | 50-200 | 0.3 | 0.3 | 0.25 |
| V21A-3 | 8-32 | 100-200 | 0.5 | 0.5 | 0.25 |
| V21A-4 | 16-64 | 100-200 | 0.3 | 0.3 | 0.25 |
| V21A-5 | 1 | 100-200 | <0.13 | <0.13 | 0.25 |
| V21A-6 | 1-2 | 100-200 | 0.3 | 0.3 | 0.25 |
| V21A-7 | 0.5-2 | 100-200 | 0.5 | 0.5 | 0.13 |
| V21A-8 | 1-2 | 100-200 | <0.13 | <0.13 | 0.25 |
| V21A-9 | 16-32 | 100-200 | 0.3 | 0.3 | 0.25 |
| V21A-10 | 16-64 | 100-200 | 0.3 | 0.3 | 0.25 |
| V21B-1 | 64 | >200 | 0.5 | 0.5 | 0.5 |
| V21B-2 | 16-64 | >200 | 0.3 | 0.5 | 0.25 |
| V21B-3 | 64 | >200 | 0.5 | 0.5 | 0.25 |
| V21B-4 | 32-64 | 200 | 0.5 | 0.5 | 0.25 |
| V21B-5 | 1-4 | >200 | 0.5 | 0.5 | 0.5 |
| V21B-6 | 16-64 | >200 | 0.5 | 0.5 | 0.25 |
| V21B-7 | 16-64 | >200 | 0.3 | 0.5 | 0.25 |
| V21B-8 | 16-64 | >200 | 0.5 | 0.5 | 0.25 |

The Minimum Inhibitory Concentration (MIC) values provided in the table above indicate that all the clindamycin, erythromycin resistant strains are susceptible to fluoroquinolones levofloxacin, nadifloxacin and besifloxacin.

### Example 5

### Animal Study with Besitfloxacin Topical Formulation

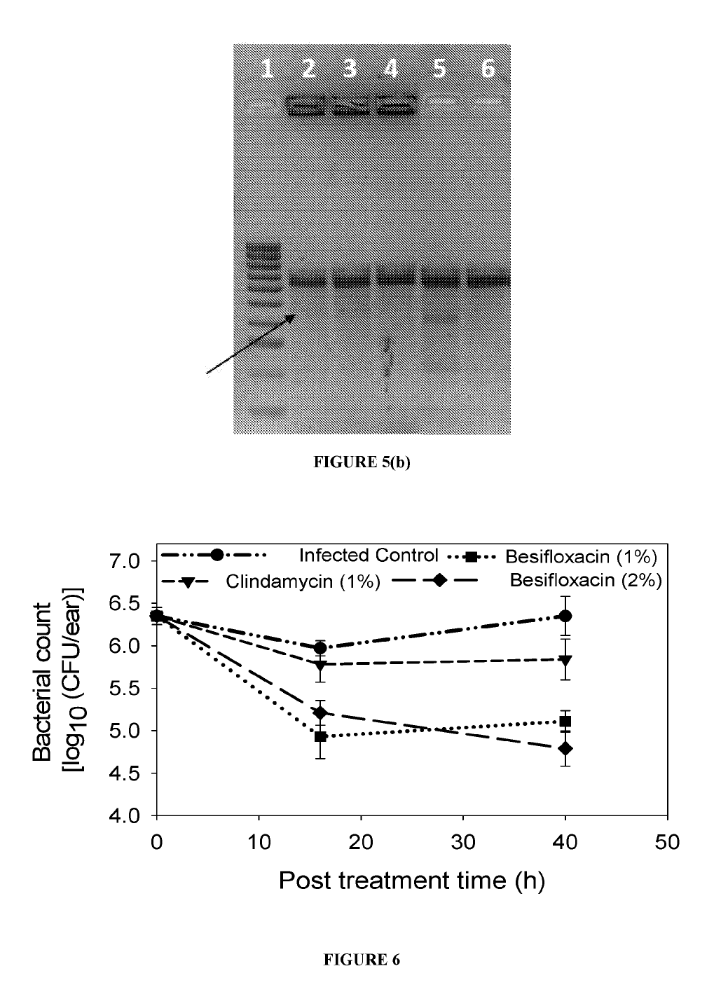

To test the in vivo efficacy of besifloxacin formulation against clindamycin resistant P. acnes, a murine ear infection model is used wherein the infection is induced using a strain of P. acnes that shows clindamycin resistance (CCARM 9010). Live P. acnes (1×107 CFU) are injected intra-dermally into mouse ears and bacterial load achieved is measured at 8 hours' post-injection. Topical treatment with besifloxacin 1% or 2% gel, or a reference treatment of clindamycin 1% gel is performed every 12 hours starting at 8 hours' post-infection. The results are graphically represented in FIG. 6, wherein the data is represented as mean ±SD. **P<0.01; ***P<0.001 when compared with infected control of the corresponding time point. As can be observed, both besifloxacin formulations (1% and 2%) impart a significant reduction in P. acnes count after about 40 hours from the initiation of treatment. This is significantly greater than the reduction in P. acnes load achieved with the use of clindamycin gel (1%) at the same time point under similar experimental conditions.

### Example 6

### Treatment of Patient Based on the Detection of Mutation in 23S rRNA Sequence of P. acnes

Acne lesions of moderate to severely affected acne patient (who did not respond to Clindamycin antibiotic treatment) are collected and the P. acnes genomic DNA present in the sample is subjected to PCR amplification using primers of SEQ ID Nos. 1 and 2 as described in Example 1, followed by detection of A2058G mutation as described in Examples 2 and 3. The genomic sequence of 23S rRNA sequence of the P. acnes from the patient is confirmed to carry the A2058G mutation.

Based on the mutation analysis, the patient is treated with about 1.00 mg of oral formulation of minocycline a day, about 0.1% of topical formulation of adapalene at night and about 1% of nadifloxacin gel in the morning. After 4 weeks of therapy, a significant improvement in the clinical lesions is observed, showing the success of the revised antibiotic treatment.

Additional embodiments and features of the present invention will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments fully reveals the general nature of the embodiments herein that others can, by applying current knowledge, readily modify and/or adapt for various applications of such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this invention have been described in terms of preferred embodiments, those of skill in the art will recognize that the embodiments herein can be practiced with modifications within the spirit and scope of the embodiments described herein.

Throughout the specification, the word “comprise”, or variations such as “comprises” or “comprising” wherever used, will be understood to imply the inclusion of a stated element, integer or step, or group of elements, integers or steps, but not the exclusion of any other element, integer or step, or group of elements, integers or steps.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the invention. These and other modifications in the nature of the invention or the preferred embodiments will be apparent to those skilled in the art from the disclosure to herein, whereby it is to be distinctly understood that the foregoing descriptive matter is to be interpreted merely as illustrative of the invention and not as a limitation. It is further understood that the scope of the present invention fully encompasses other embodiments that may become obvious to those skilled in the art and that the scope of the present invention is accordingly limited by nothing other than the appended claims.

1-29. (canceled)

30. A method for detecting Propionibacterium acnes (P. acnes) in a sample, said method comprising contacting the sample with forward primer set forth in SEQ ID No. 1 and reverse primer set forth in SEQ ID No. 2 or with forward primer set forth in SEQ ID No. 7 and reverse primer set forth in SEQ ID No. 8 and subjecting to polymerase chain reaction (PCR) to obtain an amplified product, wherein obtaining the amplified product confirms presence of P. acnes in the sample.

31. The method as claimed in claim 30, wherein the primers specifically amplify DNA encoding 23S rRNA sequence of P. acnes set forth SEQ ID No. 3, SEQ ID No. 4, SEQ ID No. 5 or SEQ ID No. 6:
wherein the SEQ ID No. 4 possesses mutation at position 2058 (E. coli numbering) with respect to wild type SEQ ID No. 3 and the mutation is A2058G; wherein the SEQ ID No. 5 possesses mutation at position 2059 (E. coli numbering) with respect to wild type SEQ ID No. 3 and the mutation is A2059 G; and wherein the SEQ ID No. 6 possesses mutation at positions 2058 and 2059 (E. coli numbering) with respect to wild type SEQ ID No. 3 and the mutations are A2058G and A2059G;
wherein the sample is selected from group comprising water, food, environmental sample and biological sample; wherein the biological sample is selected from group comprising body fluid, tissue, skin, acne lesion and bone, or any combination thereof, preferably acne lesion; wherein the body fluid is blood;
wherein the PCR amplification is carried out in presence of amplification reagent selected from group comprising dNTP, magnesium chloride, buffer, nuclease free water and DNA polymerase, or any combination thereof; wherein the P. acnes is not isolated from the sample for detection and wherein determination of the amplified product is carried out using Agarose gel electrophoresis.

32. A method for detecting mutation in 23S rRNA sequence of P. acnes, said method comprising acts of:
(a) contacting DNA encoding the rRNA sequence of P. acnes with primers specific for P. acnes 23S rRNA sequence, wherein forward primer is set forth in SEQ ID No. 1 and reverse primer is set forth in SEQ ID No. 2, or wherein forward primer is set forth in SEQ ID No. 7 and reverse primer is set forth in SEQ ID No.8;
(b) subjecting the contacted sequence of P. acnes to polymerase chain reaction (PCR) to obtain amplified product; and
(c) subjecting the amplified product to restriction enzyme digestion, or DNA hybridization followed by endonuclease digestion for detecting the mutation.

33. The method as claimed in claim 32, wherein the PCR amplification is carried out in presence of amplification reagent selected from group comprising dNTP, magnesium chloride, buffer, nuclease free water and DNA polymerase, or any combination thereof.

34. The method as claimed in claim 32, wherein the detection of mutation by restriction enzyme digestion comprises acts of:
(a) contacting the amplified product with restriction enzyme having affinity for the mutation to be detected, to enable cleaving of the amplified product at point of mutation;
(b) detecting the mutation by determining presence of cleaved amplified product post subjecting the amplified product to restriction enzyme digestion;
wherein We restriction enzyme is BpuJI and wherein the determination of the cleaved amplified product is carried out using Agarose gel electrophoresis.

35. The method as claimed in claim 32, wherein the detection of mutation by DNA hybridization followed by endonuclease digestion comprises acts of:
(a) contacting one of the amplified products with DNA encoding 23S rRNA sequence of wild type P. acnes or with DNA encoding 23S rRNA sequence of P. acnes containing the specific mutation to be detected, or contacting the amplified products individually with both; and subjecting to hybridization to obtain a duplex;
(b) contacting the duplex with a DNA endonuclease; and
(c) detecting the mutation by determining presence or absence of cleaved duplex at point of mutation, post subjecting the duplex to endonuclease digestion.

36. The method as claimed in claim 35, wherein when the amplified product has a mutation, hybridization with the DNA encoding 23S rRNA sequence of wild type P. acnes results in a cleaved duplex post contacting with the DNA endonuclease; when the amplified product has a mutation, hybridization with the DNA encoding 23S rRNA sequence of P. acnes containing the specific mutation results in an un-cleaved duplex post contacting with the DNA endonuclease; or when the amplified product has no mutation, hybridization with the DNA encoding 23S rRNA sequence of wild type P. acnes results in an un-cleaved duplex post contacting with the DNA endonuclease; or when the amplified product has no mutation or a mutation other than the mutation present in the sequence it is hybridized with, hybridization with the DNA encoding 23S rRNA sequence of P. acnes containing the specific mutation results in a cleaved duplex post contacting with the DNA endonuclease; wherein the DNA hybridization is carried out by melting the contacted amplified product at temperature ranging from about 90° C. to about 95° C. for time period ranging from about 5 to about 10 minutes to obtain melted strands, followed by reannealing the melted strands by cooling to a temperature ranging from about 20° C. to about 27° C. to obtain the duplex; and wherein the DNA endonuclease is a mismatch specific endonuclease selected from group comprising CELII, CELI and T7 endonuclease; and
wherein the determination of the cleaved duplex is carried out using Agarose gel electrophoresis.

37. The method as claimed in claim 32, wherein the P. acnes is present in a sample selected from group comprising water, food, environmental sample and biological sample;
wherein the mutation in the 23S rRNA of P. acnes is selected from A2058G (E. coli numbering), A2059G (E. coli numbering) or a combination thereof, preferably A2058G (E. coli numbering);
wherein when the mutation is A2058G or a combination of A2058G and A2059G, the P. acnes is rendered antibiotic resistant; wherein the P. acnes is not isolated from the sample for detection; wherein the sample is a biological sample selected from group comprising body fluid, tissue, skin, acne lesion and bone, or any combination thereof, preferably acne lesion; and wherein the body fluid is blood.

38. A method of treating acne in a patient infected with P. acnes, said method comprising acts of:
(a) obtaining sample from a patient;
(b) detecting presence or absence of mutation in P. acnes by the method as claimed in claim 5 to determine whether said P. acnes is resistant or sensitive to an antibiotic A; and
(c) administering the patient with an antibiotic B if the P. acnes is resistant to the antibiotic A.

39. The method as claimed in claim 38, wherein the P. acnes is not isolated from the sample for detection; and
wherein the sample is a biological sample selected from group comprising body fluid, tissue, skin, acne lesion and bone, or any combination thereof, preferably acne lesion; and wherein the body fluid is blood.

40. The method as claimed in claim 37, wherein the antibiotic is lincosamide or macrolide or a combination thereof, preferably clindamycin, erythromycin or a combination thereof.

41. The method as claimed in claim 38, wherein the antibiotic A is lincosamide or macrolide or a combination thereof, preferably clindamycin, erythromycin or a combination thereof; and wherein the antibiotic B is a fluoroquinolone antibiotic selected from group comprising besifloxacin, nadifloxacin and levofloxacin;
wherein the antibiotic B is present at concentration ranging from about 0.1% w/w to about 10% w/w, preferably from about 0.2% w/w to about 5% w/w and more preferably from about 1% w/w to about 2% w/w; and wherein the antibiotic B is in form of a formulation selected from group comprising topical formulation, solid oral formulation, liquid oral formulation, inhalation formulation, nasal formulation, ophthalmic formulation, parenteral formulation, phytoceutical, nutraceutical and food stuff or any combination thereof, preferably topical formulation;
wherein the topical formulation is selected from group comprising cream, gel, spray, foam, lotion or any combination thereof; the solid oral formulation is selected from group comprising tablet, capsule, troche, lozenge, dispersible powder, dispersible granule or any combination thereof; the liquid oral formulation is selected from group comprising aqueous or oily suspension, emulsion, drop, emulsion in hard or soft gel capsule, syrup, elixir or any combination thereof; the parenteral formulation is selected from group comprising intravenous injection, intramuscular injection, intramuscular depot, subcutaneous injection, percutaneous injection or any combination thereof; the inhalation formulation is selected from group comprising inhaler, dry powder inhaler, nebulizer or any combination thereof; the nasal formulation is selected from group comprising nasal drops, nasal sprays or a combination thereof; and the ophthalmic formulation comprises eye drops;
and wherein the formulation further comprises tetracycline, adapalene, benzoyl peroxide, or combinations thereof.

42. A kit for detecting and optionally treating P. acnes, said kit comprising primer sequences of SEQ ID Nos. 1 and 2 or primer sequences of SEQ ID Nos. 7 and 8, or both, optionally along with dNTPs, DNA polymerase, buffer, nuclease free water, magnesium chloride, vials, restriction endonuclease, DNA endonuclease, P. acnes wild type strain, DNA encoding 23S rRNA of P. acnes containing A2058G mutation, DNA encoding 23S rRNA of P. acnes containing A2059G mutation, DNA encoding 23S rRNA of P. acnes containing A2058G and A2059G mutation, instruction manual, and an antibiotic, or any combination thereof.

43. The kit as claimed in claim 42, wherein the antibiotic is fluoroquinolone, optionally along with antibiotic selected from group comprising lincosamide, macrolide, retinoid, tetracycline and benzoyl peroxide, or any combination thereof;
wherein the fluoroquinolone antibiotic is selected from group comprising besifloxacin, nadifloxacin and levofloxacin, or combinations thereof; wherein the lincosamide is clindamycin; wherein the macrolide is erythromycin; and wherein the retinoid is adapalene; wherein the antibiotic is present at concentration ranging from about 0.1% w/w to about 10% w/w, preferably from about 0.2% w/w to about 5% w/w and more preferably from about 1% w/w to about 2% w/w; and wherein the antibiotic is in form of a formulation selected from group comprising topical formulation, solid oral formulation, liquid oral formulation, inhalation formulation, nasal formulation, ophthalmic formulation, parenteral formulation, phytoceutical, nutraceutical and food stuff or any combination thereof, preferably topical formulation; and wherein the topical formulation is selected from group comprising cream, gel spray, foam, lotion or any combination thereof; the solid oral formulation is selected from group comprising tablet, capsule, troche, lozenge, dispersible powder, dispersible granule or any combination thereof; the liquid oral formulation is selected from group comprising aqueous or oily suspension, emulsion, drop, emulsion in hard or soft gel capsule, syrup, elixir or any combination thereof; the parenteral formulation is selected from group comprising intravenous injection, intramuscular injection, intramuscular depot, subcutaneous injection, percutaneous injection or any combination thereof; the inhalation formulation is selected from group comprising inhaler, dry powder inhaler, nebulizer or any combination thereof; the nasal formulation is selected from group comprising nasal drops, nasal sprays or a combination thereof; and the ophthalmic formulation comprises eye drops.
The kit as claimed in claim 13, wherein the kit is used for detecting mutation in 23S rRNA of P. acnes, wherein the mutation is selected from A2058G (E. coli numbering), A2059G (E. coli numbering) or a combination thereof with respect to wild type P. acnes, preferably A2058G (E. coli numbering); wherein the restriction endonuclease is BpuJI; and wherein the DNA endonuclease is a mis-match specific DNA endonuclease selected from group comprising CELII, CELI and T7 endonuclease; and
wherein when the mutation detected is A2058G or a combination of A2058G and A2059G, the P. acnes detected is rendered resistant to antibiotic selected from lincosamide and macrolide or a combination thereof, preferably clindamycin, erythromycin or a combination thereof.


SEQUENCE LISTING (8 sequences)

SEQ ID NO: 1 - DNA, 20 bp, Propionibacterium acnes
  primer_bind (1)..(20): Forward primer
ctgtgagtgtgatgcgtagc

SEQ ID NO: 2 - DNA, 20 bp, Propionibacterium acnes
  primer_bind (1)..(20): Reverse primer
acatcgaggtgccaaaccat

SEQ ID NO: 3 - DNA, 646 bp, Propionibacterium acnes
  gene (1)..(646): Wild type 23S rRNA
ctgtgagtgtgatgcgtagcggtgaacttaagccccagtaaacggcggtggtaactataaccatcctaaggtagcgaaattccttgtcgggtaagttccgacctgcacgaatggagtaacgatttccctactgtctccaccatgaactcggtgaaattgcattacgagtaaagatgctcgttacgcgcagcaggacggaaagaccccgggacctttactatagtttggtattggtgattgggacggtttgtgtaggataggtgggagacgttgatgcggccacgctagtggtggtggagtcgttggtgaaataccactctgattgttctggttatctcactgtggaccgtgatccggttcaaggacagtgcctgatgggtagtttgactggggcggtcgcctcctaaagggtaacggaggcgcccaaaggttccctcatcctggttggtaatcaggtggtgagtgtaagtgcacaagggggcttgactgtgagactgacaggtcgagcagggacgaaagtcgggactagtgatctgacggtggcttgtggatgcgccgtcactcaacggataaaaggtaccccggggataacaggctgatcttgcccgagcgctcatagcgacggcatggtttggcacctcgatgt

SEQ ID NO: 4 - DNA, 646 bp, Propionibacterium acnes
  mutation (1)..(646): Mutated 23S rRNA (A2058G)
ctgtgagtgtgatgcgtagcggtgaacttaagccccagtaaacggcggtggtaactataaccatcctaaggtagcgaaattccttgtcgggtaagttccgacctgcacgaatggagtaacgatttccctactgtctccaccatgaactcggtgaaattgcattacgagtaaagatgctcgttacgcgcagcaggacgggaagaccccgggacctttactatagtttggtattggtgattgggacggtttgtgtaggataggtgggagacgttgatgcggccacgctagtggtggtggagtcgttggtgaaataccactctgattgttctggttatctcactgtggaccgtgatccggttcaaggacagtgcctgatgggtagtttgactggggcggtcgcctcctaaagggtaacggaggcgcccaaaggttccctcatcctggttggtaatcaggtggtgagtgtaagtgcacaagggggcttgactgtgagactgacaggtcgagcagggacgaaagtcgggactagtgatctgacggtggcttgtggatgcgccgtcactcaacggataaaaggtaccccggggataacaggctgatcttgcccgagcgctcatagcgacggcatggtttggcacctcgatgt

SEQ ID NO: 5 - DNA, 646 bp, Propionibacterium acnes
  mutation (1)..(646): Mutated 23S rRNA (A2059G)
ctgtgagtgtgatgcgtagcggtgaacttaagccccagtaaacggcggtggtaactataaccatcctaaggtagcgaaattccttgtcgggtaagttccgacctgcacgaatggagtaacgatttccctactgtctccaccatgaactcggtgaaattgcattacgagtaaagatgctcgttacgcgcagcaggacggagagaccccgggacctttactatagtttggtattggtgattgggacggtttgtgtaggataggtgggagacgttgatgcggccacgctagtggtggtggagtcgttggtgaaataccactctgattgttctggttatctcactgtggaccgtgatccggttcaaggacagtgcctgatgggtagtttgactggggcggtcgcctcctaaagggtaacggaggcgcccaaaggttccctcatcctggttggtaatcaggtggtgagtgtaagtgcacaagggggcttgactgtgagactgacaggtcgagcagggacgaaagtcgggactagtgatctgacggtggcttgtggatgcgccgtcactcaacggataaaaggtaccccggggataacaggctgatcttgcccgagcgctcatagcgacggcatggtttggcacctcgatgt

SEQ ID NO: 6 - DNA, 646 bp, Propionibacterium acnes
  mutation (1)..(646): Mutated 23S rRNA (A2058G, A2059G)
ctgtgagtgtgatgcgtagcggtgaacttaagccccagtaaacggcggtggtaactataaccatcctaaggtagcgaaattccttgtcgggtaagttccgacctgcacgaatggagtaacgatttccctactgtctccaccatgaactcggtgaaattgcattacgagtaaagatgctcgttacgcgcagcaggacggggagaccccgggacctttactatagtttggtattggtgattgggacggtttgtgtaggataggtgggagacgttgatgcggccacgctagtggtggtggagtcgttggtgaaataccactctgattgttctggttatctcactgtggaccgtgatccggttcaaggacagtgcctgatgggtagtttgactggggcggtcgcctcctaaagggtaacggaggcgcccaaaggttccctcatcctggttggtaatcaggtggtgagtgtaagtgcacaagggggcttgactgtgagactgacaggtcgagcagggacgaaagtcgggactagtgatctgacggtggcttgtggatgcgccgtcactcaacggataaaaggtaccccggggataacaggctgatcttgcccgagcgctcatagcgacggcatggtttggcacctcgatgt

SEQ ID NO: 7 - DNA, 20 bp, Propionibacterium acnes
  primer_bind (1)..(20): Forward primer
gacactcacactacgcatcg

SEQ ID NO: 8 - DNA, 20 bp, Propionibacterium acnes
  primer_bind (1)..(20): Reverse primer
tgtagctccacggtttggta